(19) United States
(12) Patent Application Publication
(10) Pub. No.: US 2009/0054749 A1
(43) Pub. Date: Feb. 26, 2009

(54) Method and System for Providing Data Transmission in a Data Management System

(75) Inventor: Lei He, Moraga, CA (US)

Correspondence Address: JACKSON & CO., LLP, 6114 LA SALLE AVENUE, #507, OAKLAND, CA, 94611-2802, US

(73) Assignee: Abbott Diabetes Care, Inc., Alameda, CA (US)

(21) Appl. No.: 11/421,441

(22) Filed: May 31, 2006

(51) Int. Cl. A61B 5/1477

(52) U.S. Cl. 600/316; 343/720

(57) ABSTRACT

Methods and apparatuses for providing a data transmission unit antenna for wireless data transmission in a data monitoring and management system are provided.

### BACKGROUND

Analyte, e.g., glucose monitoring systems including continuous and discrete monitoring systems generally include a battery powered and microprocessor controlled system which is configured to detect signals proportional to the corresponding measured glucose levels using an electrometer, and RF signals to transmit the collected data. One aspect of certain glucose monitoring systems include a transcutaneous or subcutaneous analyte sensor configuration which is, for example, partially mounted on the skin of a subject whose glucose level is to be monitored. The sensor may use a two or three-electrode (work, reference and counter electrodes) configuration driven by a controlled potential (potentiostat) analog circuit connected through a contact system.

The analyte sensor may be configured so that at least a portion thereof is placed under the skin of the patient so as to detect the analyte levels of the patient, and another portion of segment of the analyte sensor that is in communication with the transmitter unit. The transmitter unit is configured to transmit the analyte levels detected by the sensor over a wireless communication link such as an RF (radio frequency) communication link.

In a typical configuration, the transmitter unit coupled to the analyte sensor is positioned on the skin of the patient. As such, there is an increasing desire to reduce the physical size of the on-body transmitter unit so as to minimize potentially hindering the patient's movement or daily activities, while increasing the level of comfort in wearing such on-body devices. With the increasing reduction in the size of the transmitter unit, the transmitter antenna size has been decreasing so as to fit within the housing of the transmitter unit. With the reduction in size, however, the antenna efficiency and gain generally degrades. In addition, the body of the patient which is in contact with the transmitter unit also adversely effects the performance of the antenna, potentially increasing the likelihood of data drop off and/or loss link in low signal areas.

Smaller on-body wireless devices commonly use radio frequency bands of approximately 400 MHz to transmit and/or receive data as the patient's body attenuates electromagnetic signals in proportion to the radio frequency in the direction behind the human body—that is, human body creates a radio emission shadow, and absorbs radio frequency energy. At 400 MHz frequency bands, the ideal dipole antenna length is approximately 375 mm, which is about half of the waive length in air. However, it is generally impractical to mount such a lengthy antenna on the patient's body for wireless data transmission.

In view of the foregoing, it would be desirable to have an approach to provide an antenna configuration of a data transmission device which may be configured for small compact data transmission devices while maintaining or improving its performance so as to not result in data drop-off or loss of data link in low signal areas.

### SUMMARY OF THE INVENTION

In view of the foregoing, in accordance with the various embodiments of the present invention, there is provided a method and apparatus for providing a transmitter unit antenna configuration including a plurality of inductors and capacitors for use in the data monitoring and management system.

These and other objects, features and advantages of the present invention will become more fully apparent from the following detailed description of the embodiments, the appended claims and the accompanying drawings.

### BRIEF DESCRIPTION OF THE DRAWINGS

FIG. 1 illustrates a block diagram of a data monitoring and management system for practicing one embodiment of the present invention;

FIG. 2 is a block diagram of the transmitter unit of the data monitoring and management system shown in FIG. 1 in accordance with one embodiment of the present invention;

FIG. 3 is a block diagram of the receiver/monitor unit of the data monitoring and management system shown in FIG. 1 in accordance with one embodiment of the present invention;

FIGS. 4A-4B illustrate a vertically and horizontally oriented inductor, respectively for use in the antenna configuration of the transmitter unit in accordance with one embodiment of the present invention;

FIG. 5 is a circuit schematic for illustrating a multiple-inductor antenna configuration coupled a tuning capacitor section in accordance with one embodiment of the present invention; and

FIG. 6 illustrates a multiple inductor antenna for use in the transmitter unit of the data monitoring and management system in accordance with one embodiment of the present invention.

### DETAILED DESCRIPTION

As described in accordance with the various embodiments of the present invention below, there are provided methods and system for an antenna configuration including a plurality of inductors operatively coupled to a resonant frequency tuning section in an electronic device such as a data transmitter unit used in data monitoring and management systems such as, for example, in glucose monitoring and management systems.

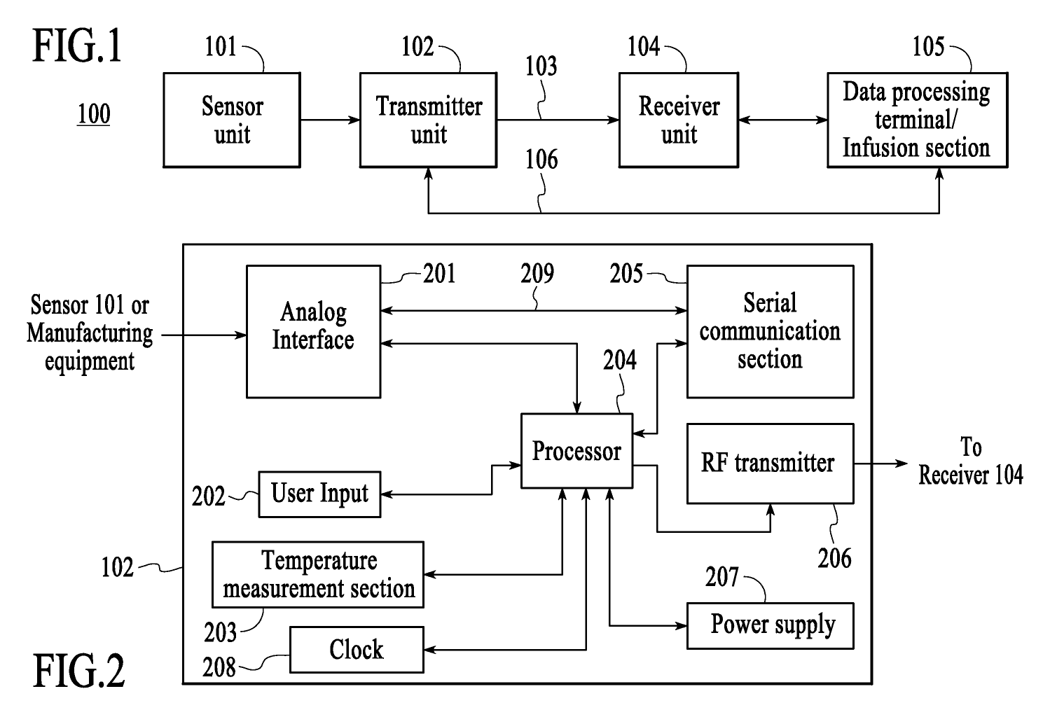

More specifically, FIG. 1 illustrates a data monitoring and management system such as, for example, analyte (e.g., glucose) monitoring system 100 in accordance with one embodiment of the present invention. The subject invention is further described primarily with respect to a glucose monitoring system for convenience and such description is in no way intended to limit the scope of the invention. It is to be understood that the analyte monitoring system may be configured to monitor a variety of analytes, e.g., lactate, and the like.

Analytes that may be monitored include, for example, acetyl choline, amylase, bilirubin, cholesterol, chorionic gonadotropin, creatine kinase (e.g., CK-MB), creatine, DNA, fructosamine, glucose, glutamine, growth hormones, hormones, ketones, lactate, peroxide, prostate-specific antigen, prothrombin, RNA, thyroid stimulating hormone, and troponin. The concentration of drugs, such as, for example, antibiotics (e.g., gentamicin, vancomycin, and the like), digitoxin, digoxin, drugs of abuse, theophylline, and warfarin, may also be monitored.

The analyte monitoring system 100 includes a sensor 101, a transmitter unit 102 coupled to the sensor 101, and a receiver unit 104 which is configured to communicate with the transmitter unit 102 via a communication link 103. The receiver unit 104 may be further configured to transmit data to a data processing terminal 105 for evaluating the data received by the receiver unit 104. Moreover, the data processing terminal in one embodiment may be configured to receive data directly from the transmitter unit 102 via a communication link 106 which may optionally be configured for bi-directional communication.

Only one sensor 101, transmitter unit 102, receiver unit 104, communication link 103, and data processing terminal 105 are shown in the embodiment of the analyte monitoring system 100 illustrated in FIG. 1. However, it will be appreciated by one of ordinary skill in the art that the analyte monitoring system 100 may include one or more sensor 101, transmitter unit 102, receiver unit 104, communication link 103, and data processing terminal 105. Moreover, within the scope of the present invention, the analyte monitoring system 100 may be a continuous monitoring system, or semi-continuous, or a discrete monitoring system. In a multi-component environment, each device is configured to be uniquely identified by each of the other devices in the system so that communication conflict is readily resolved between the various components within the analyte monitoring system 100.

In one embodiment of the present invention, the sensor 101 is physically positioned in or on the body of a user whose analyte level is being monitored. The sensor 101 may be configured to continuously sample the analyte level of the user and convert the sampled analyte level into a corresponding data signal for transmission by the transmitter unit 102. In one embodiment, the transmitter unit 102 is mounted on the sensor 101 so that both devices are positioned on the user's body. The transmitter unit 102 performs data processing such as filtering and encoding on data signals, each of which corresponds to a sampled analyte level of the user, for transmission to the receiver unit 104 via the communication link 103.

In one embodiment, the analyte monitoring system 100 is configured as a one-way RF communication path from the transmitter unit 102 to the receiver unit 104. In such embodiment, the transmitter unit 102 transmits the sampled data signals received from the sensor 101 without acknowledgement from the receiver unit 104 that the transmitted sampled data signals have been received. For example, the transmitter unit 102 may be configured to transmit the encoded sampled data signals at a fixed rate (e.g., at one minute intervals) after the completion of the initial power on procedure. Likewise, the receiver unit 104 may be configured to detect such transmitted encoded sampled data signals at predetermined time intervals. Alternatively, the analyte monitoring system 100 may be configured with a bi-directional RF (or otherwise) communication between the transmitter unit 102 and the receiver unit 104.

Additionally, in one aspect, the receiver unit 104 may include two sections. The first section is an analog interface section that is configured to communicate with the transmitter unit 102 via the communication link 103. In one embodiment, the analog interface section may include an RF receiver and an antenna for receiving and amplifying the data signals from the transmitter unit 102, which are thereafter, demodulated with a local oscillator and filtered through a band-pass filter. The second section of the receiver unit 104 is a data processing section which is configured to process the data signals received from the transmitter unit 102 such as by performing data decoding, error detection and correction, data clock generation, and data bit recovery.

In operation, upon completing the power-on procedure, the receiver unit 104 is configured to detect the presence of the transmitter unit 102 within its range based on, for example, the strength of the detected data signals received from the transmitter unit 102 or a predetermined transmitter identification information. Upon successful synchronization with the corresponding transmitter unit 102, the receiver unit 104 is configured to begin receiving from the transmitter unit 102 data signals corresponding to the user's detected analyte level. More specifically, the receiver unit 104 in one embodiment is configured to perform synchronized time hopping with the corresponding synchronized transmitter unit 102 via the communication link 103 to obtain the user's detected analyte level.

Referring again to FIG. 1, the data processing terminal 105 may include a personal computer, a portable computer such as a laptop or a handheld device (e.g., personal digital assistants (PDAs)), and the like, each of which may be configured for data communication with the receiver via a wired or a wireless connection. Additionally, the data processing terminal 105 may further be connected to a data network (not shown) for storing, retrieving and updating data corresponding to the detected analyte level of the user.

Within the scope of the present invention, the data processing terminal 105 may include an infusion device such as an insulin infusion pump or the like, which may be configured to administer insulin to patients, and which may be configured to communicate with the receiver unit 104 for receiving, among others, the measured analyte level. Alternatively, the receiver unit 104 may be configured to integrate an infusion device therein so that the receiver unit 104 is configured to administer insulin therapy to patients, for example, for administering and modifying basal profiles, as well as for determining appropriate boluses for administration based on, among others, the detected analyte levels received from the transmitter unit 102.

Additionally, the transmitter unit 102, the receiver unit 104 and the data processing terminal 105 may each be configured for bi-directional wireless communication such that each of the transmitter unit 102, the receiver unit 104 and the data processing terminal 105 may be configured to communicate (that is, transmit data to and receive data from) with each other via the wireless communication link 103. More specifically, the data processing terminal 105 may in one embodiment be configured to receive data directly from the transmitter unit 102 via the communication link 106, where the communication link 106, as described above, may be configured for bi-directional communication.

In this embodiment, the data processing terminal 105 which may include an insulin pump, may be configured to receive the analyte signals from the transmitter unit 102, and thus, incorporate the functions of the receiver 103 including data processing for managing the patient's insulin therapy and analyte monitoring. In one embodiment, the communication link 103 may include one or more of an RF communication protocol, an infrared communication protocol, a Bluetooth enabled communication protocol, an 802.11x wireless communication protocol, or an equivalent wireless communication protocol which would allow secure, wireless communication of several units (for example, per HIPPA requirements) while avoiding potential data collision and interference.

FIG. 2 is a block diagram of the transmitter of the data monitoring and detection system shown in FIG. 1 in accordance with one embodiment of the present invention. Referring to the Figure, the transmitter unit 102 in one embodiment includes an analog interface 201 configured to communicate with the sensor 101 (FIG. 1), a user input 202, and a temperature detection section 203, each of which is operatively coupled to a transmitter processor 204 such as a central processing unit (CPU). As can be seen from FIG. 2, there are provided four contacts, three of which are electrodes—work electrode, guard contact, reference electrode, and counter electrode, each operatively coupled to the analog interface 201 of the transmitter unit 102 for connection to the sensor unit 201 (FIG. 1). In one embodiment, each of the work electrode, guard contact, reference electrode, and counter electrode may be made using a conductive material that is either printed or etched, for example, such as carbon which may be printed, or metal foil (e.g., gold) which may be etched.

Further shown in FIG. 2 are a transmitter serial communication section 205 and an RF transmitter 206, each of which is also operatively coupled to the transmitter processor 204. Moreover, a power supply 207 such as a battery is also provided in the transmitter unit 102 to provide the necessary power for the transmitter unit 102. Additionally, as can be seen from the Figure, clock 208 is provided to, among others, supply real time information to the transmitter processor 204.

In one embodiment, a unidirectional input path is established from the sensor 101 (FIG. 1) and/or manufacturing and testing equipment to the analog interface 201 of the transmitter unit 102, while a unidirectional output is established from the output of the RF transmitter 206 of the transmitter unit 102 for transmission to the receiver unit 104. In this manner, a data path is shown in FIG. 2 between the aforementioned unidirectional input and output via a dedicated link 209 from the analog interface 201 to serial communication section 205, thereafter to the processor 204, and then to the RF transmitter 206. As such, in one embodiment, via the data path described above, the transmitter unit 102 is configured to transmit to the receiver unit 104 (FIG. 1), via the communication link 103 (FIG. 1), processed and encoded data signals received from the sensor 101 (FIG. 1). Additionally, the unidirectional communication data path between the analog interface 201 and the RF transmitter 206 discussed above allows for the configuration of the transmitter unit 102 for operation upon completion of the manufacturing process as well as for direct communication for diagnostic and testing purposes.

As discussed above, the transmitter processor 204 is configured to transmit control signals to the various sections of the transmitter unit 102 during the operation of the transmitter unit 102. In one embodiment, the transmitter processor 204 also includes a memory (not shown) for storing data such as the identification information for the transmitter unit 102, as well as the data signals received from the sensor 101. The stored information may be retrieved and processed for transmission to the receiver unit 104 under the control of the transmitter processor 204. Furthermore, the power supply 207 may include a commercially available battery.

The transmitter unit 102 is also configured such that the power supply section 207 is capable of providing power to the transmitter for a minimum of about three months of continuous operation after having been stored for about eighteen months in a low-power (non-operating) mode. In one embodiment, this may be achieved by the transmitter processor 204 operating in low power modes in the non-operating state, for example, drawing no more than approximately 1 μA of current. Indeed, in one embodiment, the final step during the manufacturing process of the transmitter unit 102 may place the transmitter unit 102 in the lower power, non-operating state (i.e., post-manufacture sleep mode). In this manner, the shelf life of the transmitter unit 102 may be significantly improved.

Moreover, as shown in FIG. 2, while the power supply unit 207 is shown as coupled to the processor 204, and as such, the processor 204 is configured to provide control of the power supply unit 207, it should be noted that within the scope of the present invention, the power supply unit 207 is configured to provide the necessary power to each of the components of the transmitter unit 102 shown in FIG. 2.

Referring back to FIG. 2, the power supply section 207 of the transmitter unit 102 in one embodiment may include a rechargeable battery unit that may be recharged by a separate power supply recharging unit (for example, provided in the receiver unit 104) so that the transmitter unit 102 may be powered for a longer period of usage time. Moreover, in one embodiment, the transmitter unit 102 may be configured without a battery in the power supply section 207, in which case the transmitter unit 102 may be configured to receive power from an external power supply source (for example, a battery) as discussed in further detail below.

Referring yet again to FIG. 2, the temperature detection section 203 of the transmitter unit 102 is configured to monitor the temperature of the skin near the sensor insertion site. The temperature reading is used to adjust the analyte readings obtained from the analog interface 201. The RF transmitter 206 of the transmitter unit 102 may be configured for operation in the frequency band of 315 MHz to 322 MHz, for example, in the United States. Further, in one embodiment, the RF transmitter 206 is configured to modulate the carrier frequency by performing Frequency Shift Keying and Manchester encoding. In one embodiment, the data transmission rate is 19,200 symbols per second, with a minimum transmission range for communication with the receiver unit 104.

Additional detailed description of the continuous analyte monitoring system, its various components including the functional descriptions of the transmitter are provided in U.S. Pat. No. 6,175,752 issued Jan. 16, 2001 entitled “Analyte Monitoring Device and Methods of Use”, and in application Ser. No. 10/745,878 filed Dec. 26, 2003 entitled “Continuous Glucose Monitoring System and Methods of Use”, each assigned to the Assignee of the present application.

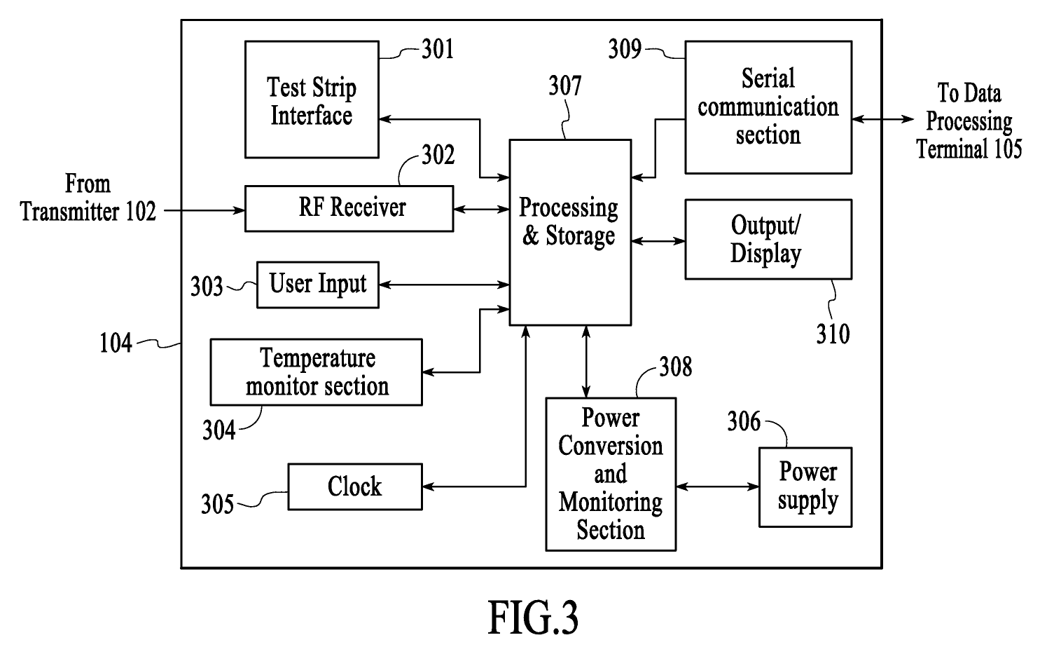

FIG. 3 is a block diagram of the receiver/monitor unit of the data monitoring and management system shown in FIG. 1 in accordance with one embodiment of the present invention. Referring to FIG. 3, the receiver unit 104 includes a blood glucose 25 test strip interface 301, an RF receiver 302, an input 303, a temperature detection section 304, and a clock 305, each of which is operatively coupled to a receiver processor 307. As can be further seen from the Figure, the receiver unit 104 also includes a power supply 306 operatively coupled to a power conversion and monitoring section 308. Further, the power conversion and monitoring section 308 is 30 also coupled to the receiver processor 307. Moreover, also shown are a receiver serial communication section 309, and an output 310, each operatively coupled to the receiver processor 307.

In one embodiment, the test strip interface 301 includes a glucose level testing portion to receive a manual insertion of a glucose test strip, and thereby determine and display the glucose level of the test strip on the output 310 of the receiver unit 104. This manual testing of glucose can be used to calibrate sensor 101. The RF receiver 302 is configured to communicate, via the communication link 103 (FIG. 1) with the RF transmitter 206 of the transmitter unit 102, to receive encoded data signals from the transmitter unit 102 for, among others, signal mixing, demodulation, and other data processing. The input 303 of the receiver unit 104 is configured to allow the user to enter information into the receiver unit 104 as needed. In one aspect, the input 303 may include one or more keys of a keypad, a touch-sensitive screen, or a voice-activated input command unit. The temperature detection section 304 is configured to provide temperature information of the receiver unit 104 to the receiver processor 307, while the clock 305 provides, among others, real time information to the receiver processor 307.

Each of the various components of the receiver unit 104 shown in FIG. 3 is powered by the power supply 306 which, in one embodiment, includes a battery. Furthermore, the power conversion and monitoring section 308 is configured to monitor the power usage by the various components in the receiver unit 104 for effective power management and to alert the user, for example, in the event of power usage which renders the receiver unit 104 in sub-optimal operating conditions. An example of such sub-optimal operating condition may include, for example, operating the vibration output mode (as discussed below) for a period of time thus substantially draining the power supply 306 while the processor 307 (thus, the receiver unit 104) is turned on. Moreover, the power conversion and monitoring section 308 may additionally be configured to include a reverse polarity protection circuit such as a field effect transistor (FET) configured as a battery activated switch.

The serial communication section 309 in the receiver unit 104 is configured to provide a bi-directional communication path from the testing and/or manufacturing equipment for, among others, initialization, testing, and configuration of the receiver unit 104. Serial communication section 104 can also be used to upload data to a computer, such as time-stamped blood glucose data. The communication link with an external device (not shown) can be made, for example, by cable, infrared (IR) or RF link. The output 310 of the receiver unit 104 is configured to provide, among others, a graphical user interface (GUI) such as a liquid crystal display (LCD) for displaying information. Additionally, the output 310 may also include an integrated speaker for outputting audible signals as well as to provide vibration output as commonly found in handheld electronic devices, such as mobile telephones presently available. In a further embodiment, the receiver unit 104 also includes an electro-luminescent lamp configured to provide backlighting to the output 310 for output visual display in dark ambient surroundings.

Referring back to FIG. 3, the receiver unit 104 in one embodiment may also include a storage section such as a programmable, non-volatile memory device as part of the processor 307, or provided separately in the receiver unit 104, operatively coupled to the processor 307. The processor 307 is further configured to perform Manchester decoding as well as error detection and correction upon the encoded data signals received from the transmitter unit 102 via the communication link 103.

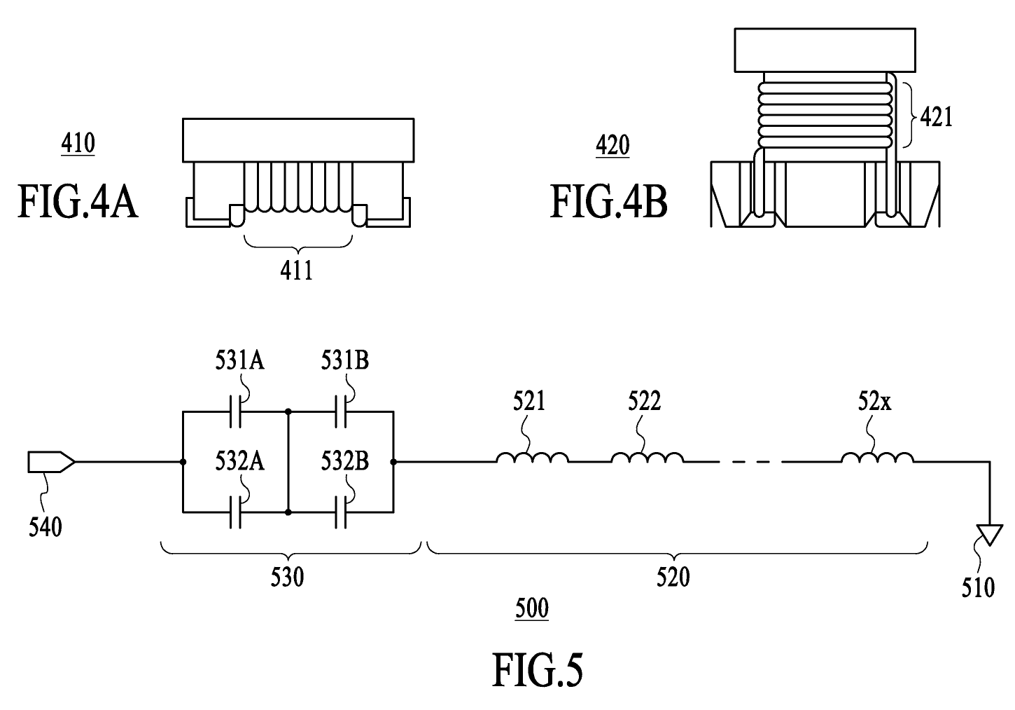

FIGS. 4A-4B illustrate a vertically and horizontally oriented inductor, respectively for use in the antenna configuration of the transmitter unit in accordance with one embodiment of the present invention. More specifically, referring to FIG. 4A, a vertically oriented inductor 410 configuration for the transmitter unit antenna, which includes a predetermined number of windings 411 is shown, while referring to FIG. 4B, a horizontally oriented inductor 420 configuration for the transmitter unit antenna which includes a predetermined number of windings 421 is shown.

As discussed in further detail below, in one embodiment of the present invention the transmitter unit antenna may be provided with a plurality of either the vertically oriented inductors 410, or the horizontally oriented inductors 420, or combinations thereof. In this manner, in one aspect of the present invention, data signal range (transmission and/or reception) for the antenna may be improved approximately ten to fifteen decibels.

In one aspect, the number of windings 411, 421 of the vertically oriented inductor or the horizontally oriented inductor may be modified to correspondingly adjust the total inductance of the antenna in the transmitter unit 102 (FIG. 1). That is, since the number of windings is directly proportional to the total antenna inductance, in one embodiment, the number of windings 411, 421 of the vertically oriented inductor or the horizontally oriented inductor may be increased to attain a higher inductance.

In addition, the inductance may also vary based on the frequency. For a given dimension of the inductors, the higher number of windings will reduce the Q factor of the inductor because the distributed parasitic capacitance between the wires as discussed in further detail below. Since the antenna in one embodiment includes a plurality of inductors, the total inductance of the antenna may also be increased by adding additional inductors in series if the physical dimensions allow for the addition. In this manner, in one embodiment of the present invention, the high Q factor of the antenna may be maintained.

FIG. 5 is a circuit schematic for illustrating a multiple inductor antenna configuration coupled a tuning capacitor section in accordance with one embodiment of the present invention. Referring to FIG. 5, in one embodiment of the present invention, the multiple inductor antenna section 520 is operatively coupled to a resonant frequency tuning section 530 which may be further coupled to the antenna input 540 as shown in FIG. 5. In addition, also shown in FIG. 5 is the RF ground terminal 510 which is coupled to the multiple inductor antenna section 520.

Referring to FIG. 5, the multiple inductor antenna 520 in one embodiment of the present invention includes a plurality of inductors 520, 522, 52X (where X denotes a predetermined alphanumeric value corresponding to the number of the inductors in the multiple inductor antenna section 520 coupled in series). The resonant frequency tuning section 530 in one embodiment includes a pair of series capacitors 531A, 531B and 532A, 532B, coupled in parallel, and to the multiple inductor antenna section 520.

In one aspect of the present invention, the multiple inductor antenna section 520 may be tuned based on changes in the capacitive values of the capacitors 531A, 531B, 532A, 532B of the resonant frequency tuning section 530. This may result in narrowing the bandwidth of the multiple inductor antenna section 520. In turn, the rejection of interference noise from the transmitter unit 102 circuitry also maintains the noise level down.

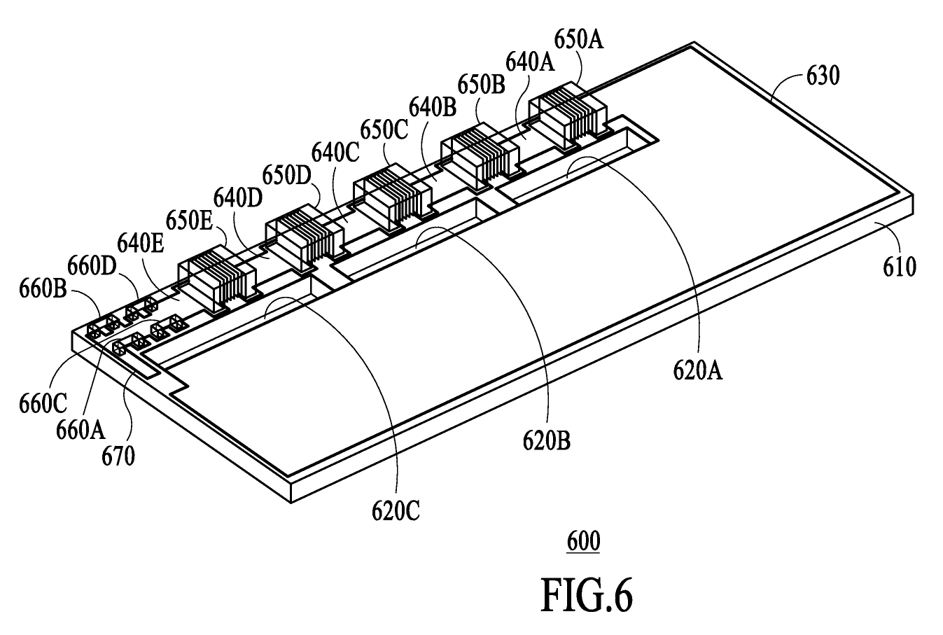

FIG. 6 illustrates a multiple inductor antenna for use in the transmitter unit of the data monitoring and management system in accordance with one embodiment of the present invention. Referring to FIG. 6, the multiple inductor antenna 600 in one embodiment of the present invention includes a dielectric substrate base 610 configured in one embodiment to provide structural support of the multiple inductor antenna 600 and its associated components including the copper trace connections, small ground platform, inductors, capacitors and other associated electronic components associated with the antenna configuration.

Referring back to FIG. 6, the multiple inductor antenna 600 in one embodiment also includes a plurality of cutout slots 620A, 620B, 620C that are provided on the dielectric substrate base 610 along the length of the antenna 600 and configured to control or minimize the effect of the dielectric material surrounding the antenna 600. That is, the dielectric substrate base 610 of the antenna 600 configuration may change the reactance characteristics of the antenna 600 based on the higher permittivity of the material properties of the dielectric substrate base 610 as compared with the permittivity of air.

In addition, sine the dielectric substrate base 610 is configured to provide structural support for the antenna 600, the parasitic capacitance distributed along the antenna 600 may be minimized by the cutout slots 620A, 620B, 620C. More specifically, the parasitic distributed capacitance is proportional to the permittivity of the media existing between the electrode terminals—which in this case are the dielectric substrate base 610, the inductors and the ground plate, and may be four times greater than the permittivity of air.

Referring again to FIG. 6, the antenna 600 may also be provided with a small ground platform 630, a plurality of copper strip traces 640A, 640B, 640C, 640D, 640E which may be printed on the dielectric substrate 610, and each configured to provide electrical connection to a corresponding one of a plurality of inductors 650A, 650B, 650C, 650D, 650E. As further shown in FIG. 6, in one embodiment, the antenna 600 may also be provided with a plurality of capacitors 660A, 660B, 660C, 660D which are configured to comprise the resonant frequency tuning section 530 (FIG. 5).

Additionally, in one embodiment of the present invention, the antenna 600 may also include an antenna feed point 670 which is electrically coupled to capacitors 660A, 660B via the copper strip trace 650E. The antenna feed point 670 in one embodiment is configured to receive and/or transmit signals. In one embodiment, the antenna feed point 670 and the small ground platform 630 form a source port with a predetermined impedance, such as, for example, 50 Ohms resistance. The source port in one embodiment is configured as the output stage of the transmitter unit 102 (FIG. 1) that electrically shares the same small ground platform 630 with the antenna 600, and is configured to deliver the output signals to the feed point 670 of the antenna 600. Alternatively, if the antenna 600 is coupled to the receiver unit 104 (FIG. 1), the antenna 600 may be configured to provide signal power through the feed point 670 to the receiver unit 104 (FIG. 1) functioning as the power source port to the receiver unit 104 (FIG. 1).

Referring yet again to FIG. 6, the plurality of capacitors 660A, 660B, 660C, 660D are electrically coupled in parallel and in series to form a predetermined capacitance, and in series with the plurality of inductors 650A, 650B, 650C, 650D, 650E to tune the antenna 600 to resonate at a predetermined frequency. When the antenna resonates at the desired frequency, the impedance of the antenna is primarily resistance without reactance contents to the feed point at the desired frequency. As such, the power will be consumed by the antenna resistance only to get maximum power delivery efficiency at desired frequency.

Further, as can be seen from FIG. 6, the plurality of inductors 650A, 650B, 650C, 650D, 650E which are electrically coupled in series may be aligned in a relatively straight line, or alternatively, in a predetermined radius curve, and configured as a radiating element of the antenna 600 of the transmitter unit 102 (FIG. 1), or a radiation detector element of the receiver antenna 600. In addition, referring back to FIG. 6, each of the plurality of inductors 650A, 650B, 650C, 650D, 650E may be configured with a horizontal winding axis on a dielectric material core, and where the winding direction of each one of the plurality of inductors 650A, 650B, 650C, 650D, 650E maybe wound in a clockwise direction or alternatively in a counterclockwise direction about the core axis. The winding of the inductors 650A, 650B, 650C, 650D, 650E create a magnetic field when there is current flowing through the inductor 650A, 650B, 650C, 650D, 650E.

The polarity of the magnetic field is determined by the winding orientation. Thus, when two opposite winding inductors are connected together and located closely, one inductor will generate a magnetic field against the other one, and the generated opposite magnetic field will reduce the current flowing in results of more resistance to the current or higher impedance. In addition, in accordance with one embodiment of the present invention, the winding direction of each of the plurality of inductors 650A, 650B, 650C, 650D, 650E may be the same, or alternatively differ. More specifically, in one embodiment, a combination of clockwise and counterclockwise winding in the plurality of inductors 650A, 650B, 650C, 650D, 650E may provide increased resistance impedance of the antenna 600 to achieve higher impedance matching circuits.

In the manner described above, in one embodiment of the present invention, the antenna 600 maybe configured to resonate at a desired or predetermined frequency band by selecting the total capacitance of the plurality of capacitors 660A, 660B, 660C, 660D, to equal to the total inductance of the plurality of inductors 650A, 650B, 650C, 650D, 650E such that maximum antenna gain and efficiency may be attained for a predetermined physical configuration of the antenna 600, and the transmitter unit dimensions. That is, the resonance of the antenna 600 occurs when the reactance portion (or imagery part) of the antenna impedance is zero at a desired frequency. The reactance includes inductive reactance and capacitive reactance. To get zero reactance, total inductive reactance (inductance) must be equal to the total capacitive reactance (capacitance). This is due to the fact that the resonant antenna at the predetermined frequency band has purity resistance impedance to simplify the matching circuit design and antenna measurement.

If the antenna 600 is not resonating at the desired frequency, the impedance of the antenna 600 may contain reactance contents. To eliminate the reactance contents of the antenna impedance, impedance of the feed point 670 towards the output stage of the transmitter unit 104 (FIG. 1) must contain opposite and equal reactance to the reactance of the antenna 600. In addition, the reactance contents of the antenna impedance will cause measurement error based on the mismatch between the antenna impedance and input impedance of test equipment because the input impedance of the equipment is purity resistance.

The antenna 600, if configured with a higher than desired Q factor, may result in instability of the antenna performance because the antenna 600 may become susceptible to the dielectric materials surrounding the antenna 600 as a result of the resonant frequency varying widely. On the other hand, the antenna 600, if configured with a lower than desired Q factor, will likely degrade the power efficiency of the transmitter unit 102 (FIG. 1) with nonlinear RF output amplifiers. Accordingly, in one embodiment of the present invention, a suitable Q factor may be obtained by modifying the ratio of the total capacitance of the plurality of capacitors 660A, 660B, 660C, 660D of the antenna 600 to the total inductance of the plurality of inductors 650A, 650B, 650C, 650D, 650E.

Moreover, the opening dimensions of the plurality of cutout slots 620A, 620B, 620C on the dielectric substrate base 610 may affect the Q factor of the antenna 600. The parasitic distributed capacitance reduces the inductance of the plurality of inductors 650A, 650B, 650C, 650D, 650E as a result of the reduction in the Q factor.

In the manner described above, in one embodiment of the present invention, there is provided an antenna for use with an on-body transmitter unit that includes a plurality of tuning capacitors coupled in series with multiple wire wound type chip inductors that are mounted on a physically small platform. The print copper traces on the board or platform may be configured to electrically connect the tuning capacitors and the multiple wire wound type chip inductors to allow a current flow through the entire antenna to radiate electromagnetic signals out of the antenna, or alternatively, to receive electromagnetic signals by the antenna.

In one aspect, a dielectric substrate material may be configured to provide structural support for the antenna, the printed copper traces, and the small ground platform as well as other electronic components associated with the transmitter unit. In addition, there may be provided in one embodiment multiple cutout slots on the dielectric material substrate between the antenna and the small ground platform, which may be configured to control the antenna dielectric loading and parasitic reactance distributed along the entire antenna.

An analyte monitoring system in accordance with one embodiment of the present invention included an analyte sensor to detect an analyte level of a patient, and a transmitter unit in signal communication with the analyte sensor; the transmitter unit further including an antenna, where the antenna includes a plurality of inductances operatively coupled in series, a ground terminal operatively coupled to one of the plurality of inductors, and a tuning circuit operatively coupled to another one of the plurality of inductors, and configured to tune the antenna to resonate at a predetermined frequency.

The tuning circuit may include a first pair of capacitances coupled in series and a second pair of capacitances coupled in series, and further the first pair of capacitances may be operatively coupled in parallel to the second pair of capacitances.

In one aspect, each of the plurality of inductances may include a predetermined number of winding.

The plurality of inductances may be configured to generate a magnetic field.

In a further aspect, the antenna may be configured to transmit or receive one or more signals.

The transmitter unit in one embodiment may be configured to be positioned on a skin of a patient.

Moreover, one or more of the analyte sensor and the transmitter unit may be implantable.

The transmitter unit in one aspect may be configured to transmit one or more signals associated with the detected analyte level of the patient.

The system in yet another aspect may include a receiver unit configured to receive the one or more signals from the transmitter unit.

A method in accordance with another embodiment of the present invention includes detecting an analyte level of a patient and providing an antenna for transmitting a signal associated with the detected analyte level, the providing step further including tuning the antenna to resonate at a predetermined frequency.

In one aspect, tuning the antenna may include operatively coupling a plurality of inductances in series, operatively coupling a ground terminal to one of the plurality of inductances, and operatively coupling a plurality of capacitances to another one of the plurality of inductors.

In addition, operatively coupling the plurality of capacitances may include coupling a first pair of capacitances in series, coupling a second pair of capacitances in series, and coupling the first pair of capacitances in parallel to the second pair of capacitances, where each of the plurality of inductances may include a predetermined number of winding.

Further, the plurality of inductances may be configured to generate a magnetic field.

The method may also include providing a housing, the housing including the antenna, and the method may additional include positioning the housing on the skin of a patient.

An apparatus in accordance with still another embodiment of the present invention includes a data transmission unit including an antenna operatively coupled to the data transmission unit, the antenna including a plurality of inductances operatively coupled in series, a ground terminal operatively coupled to one of the plurality of inductors, and a tuning circuit operatively coupled to another one of the plurality of inductors, and configured to tune the antenna to resonate at a predetermined frequency.

The tuning circuit may include a first pair of capacitances coupled in series and a second pair of capacitances coupled in series, and further where the first pair of capacitances may be operatively coupled in parallel to the second pair of capacitances.

Various other modifications and alterations in the structure and method of operation of this invention will be apparent to those skilled in the art without departing from the scope and spirit of the invention. Although the invention has been described in connection with specific preferred embodiments, it should be understood that the invention as claimed should not be unduly limited to such specific embodiments. It is intended that the following claims define the scope of the present invention and that structures and methods within the scope of these claims and their equivalents be covered thereby.

What is claimed is:

1. An analyte monitoring system, comprising:
an analyte sensor to detect an analyte level of a patient; and
a transmitter unit in signal communication with the analyte sensor; the transmitter unit further including an antenna, wherein the antenna includes:
a plurality of inductances operatively coupled in series;
a ground terminal operatively coupled to one of the plurality of inductors; and
a tuning circuit operatively coupled to another one of the plurality of inductors, and configured to tune the antenna to resonate at a predetermined frequency.

2. The system of claim 1 wherein the tuning circuit includes a first pair of capacitances coupled in series and a second pair of capacitances coupled in series, and further wherein the first pair of capacitances is further operatively coupled in parallel to the second pair of capacitances.

3. The system of claim 1 wherein each of the plurality of inductances include a predetermined number of winding.

4. The system of claim 1 wherein the plurality of inductances are configured to generate a magnetic field.

5. The system of claim 1 wherein the antenna is configured to transmit or receive one or more signals.

6. The system of claim 1 wherein the transmitter unit is configured to be positioned on a skin of a patient.

7. The system of claim 6 wherein one or more of the analyte sensor and the transmitter unit is implantable.

8. The system of claim 1 wherein the transmitter unit is configured to transmit one or more signals associated with the detected analyte level of the patient.

9. The system of claim 8 further including a receiver unit configured to receive the one or more signals from the transmitter unit.

10. A method, comprising:
detecting an analyte level of a patient; and
providing an antenna for transmitting a signal associated with the detected analyte level, the providing step further including tuning the antenna to resonate at a predetermined frequency.

11. The method of claim 10 wherein the step of tuning the antenna includes:
operatively coupling a plurality of inductances in series;
operatively coupling a ground terminal to one of the plurality of inductances; and
operatively coupling a plurality of capacitances to another one of the plurality of inductors.

12. The method of claim 11 wherein the step of operatively coupling the plurality of capacitances includes:
coupling a first pair of capacitances in series;
coupling a second pair of capacitances in series and
coupling the first pair of capacitances in parallel to the second pair of capacitances.

13. The method of claim 11 wherein each of the plurality of inductances include a predetermined number of winding.

14. The method of claim 11 wherein the plurality of inductances are configured to generate a magnetic field.

15. The method of claim 11 further including the step of providing a housing, the housing including the antenna.

16. The method of claim 15 further including the step of positioning the housing on the skin of a patient.

17. An apparatus, comprising:
a data transmission unit including an antenna operatively coupled to the data transmission unit, the antenna including:
a plurality of inductances operatively coupled in series;
a ground terminal operatively coupled to one of the plurality of inductors; and
a tuning circuit operatively coupled to another one of the plurality of inductors, and configured to tune the antenna to resonate at a predetermined frequency.

18. The apparatus of claim 17 wherein the tuning circuit includes a first pair of capacitances coupled in series and a second pair of capacitances coupled in series, and further wherein the first pair of capacitances is further operatively coupled in parallel to the second pair of capacitances.

19. The apparatus of claim 17 wherein each of the plurality of inductances include a predetermined number of winding.

20. The apparatus of claim 17 wherein the plurality of inductances are configured to generate a magnetic field.
